(19) United States
(12) Patent Application Publication
(10) Pub. No.: US 2025/0235700 A1
(43) Pub. Date: Jul. 24, 2025

(54) METHOD OF OPERATING VISUAL SENSORY DEVICE

(71) Applicant: Bionic Vision Technologies, Chatswood, New South Wales (AU)

(72) Inventors: Samuel STEFOPOULOS, Chatswood, New South Wales (AU); Xerxes BATTIWALLA, Chatswood, New South Wales (AU)

(21) Appl. No.: 18/703,601

(22) PCT Filed: Jan. 25, 2024
(86) PCT No.: PCT/AU2024/050045

§ 371 Date: Apr. 22, 2024

(30) Foreign Application Priority Data
Jan. 25, 2023 (AU) 2023900179

(51) Int. Cl. A61N 1/36; A61N 1/05

(52) U.S. Cl. CPC A61N 1/36046; A61N 1/0543

(57) ABSTRACT

A method of operating a visual sensory perception device 10 includes receiving image data and other data indicative of an externally generated image representative of environmental data. One or more predetermined objects from the image data are detected and the post-object detected image data are processed. The location of detected objects to corresponding locations of the stimulation device 10 are matched and the intensity of output is scaled to the corresponding locations of the stimulation device 10. Output data for use by the stimulation device representative of the location of the detected objects is provided.

### FIELD

The invention relates to sensory devices and, in particular, to a method of operating visual sensory devices.

The invention has been developed with respect to visual sensory input by means of retinal stimulation implant devices driven by externally acquired environmental data and will be described hereinafter with reference to this application. However, it will be appreciated that the invention is not limited to this particular field of use and is also applicable to other sensory input devices such as externally applied tactile stimulation and direct neural stimulation, for example.

### BACKGROUND

Neural stimulation by means of a visual prosthesis has been in research for many decades. Unlike the cochlear implant for neural stimulation corresponding to adjacent sound waves, visual prostheses are not widely used in patients notwithstanding significant research and expenditure. It is highly desirable to provide intraocular visual prosthesis devices that can at least partially restore some form of vision, especially in the case where a person has significant blindness that has been caused by an inherited retinal photo-receptor degenerative disease such as retinitis pigmentosa. Age-related degenerative conditions such as macular degeneration also causes significant visual impairment in many people and those sufferers lack a practical means of visually navigating their environs. Often, the only visual acuity such a person may have is where there is light or there is not.

It is currently known to implant an electrode array in the suprachoroidal space behind the eye. The array is typically in the form of a rectangular grid where columns and rows of electrodes forming the grid can be offset. The electrode array is disposed contiguously with a retina so as to provide electrical stimulation to the optic nerves. The electrode array is connected to a receiver coil that is connected to a control device external to the person where the control device provides signals used by the electrodes to provide stimulation. The control device is typically connected to the electrode array using RF communication, including for providing power to operate the electrodes.

The control device is connected to a digital imaging input source such as a charge coupled device (CCD) or photodiode array. It is well known that the size of the electrode array able to be implanted is not sufficiently large, does not have high spatial fidelity and lacks other information in natural sight such as colour, and so a direct translation of the received digital image applied the stimulation to the electrodes does not correspond to what would be understood to be a meaningful or useful image for allowing any significant autonomous functionality by the person with the degenerative eye condition. That is, the information throughput that may be able to be delivered by the electrode array is orders of magnitude lower than is capable of being provided by even relatively common digital imaging apparatus.

It is also well known that the response to particular stimulation can vary widely between people. Methods are known particularly in auditory neural stimulation to essentially tune the output of the electrodes for a given input image to provide an acceptable response in each patient. In spite of this, however, the relatively small size of the available electrode arrays for the retinal nerves makes discriminating objects detected by the digital imaging device difficult for a person when in use. As a result, there have been many efforts to attempt to detect objects and provide consistent neural stimulation to a person corresponding to those objects, but these continue to be lacking especially for very common everyday situations a blind or need blind person needs to navigate.

### OBJECT OF INVENTION

The object of the invention is a desire to provide a method of processing visual data and environmental data and translating same into corresponding neural stimulation that overcomes one or more of the disadvantages of the prior art, or to provide a useful alternative.

### SUMMARY OF INVENTION

According to a first aspect of the invention there is provided a method of operating a visual sensory substitution device, the method comprising the steps of:
receiving image data and other data indicative of an externally generated image representative of environmental data;
detecting one or more predetermined objects from the image data;
processing the post-object detected image data;
matching the location of detected objects to corresponding locations of the stimulation device;
scaling the intensity of output to the corresponding locations of the stimulation device;
providing output data to the stimulation device representative of the location of the detected objects.

According to another aspect of the invention there is provided a method of operating a visual sensory perception device, the method comprising the steps of:
receiving image data and other data indicative of an externally generated image representative of environmental data;
depth pre-processing the received image;
extracting predetermined features from the pre-processed image;
down-sampling the pre-processed image to create a corresponding intensity scaling map of the intensity of corresponding to the location stimulation device,
scaling the intensity of the pre-processed image using the intensity scaling map; and
providing output data for use by the stimulation device.

It can therefore be seen that there is advantageously provided a method of processing visual information or environmental data to assist those in need in their everyday activities by providing improved perceptible stimulation for detection of objects and processing of depth. Additionally, there is advantageously provided a method in which the input mode of the neural stimulation device can be selected to remove or include various detected objects. Furthermore, it will be understood that in addition to implanted devices such as retinal stimulation electrode arrays, the method is applicable for other sensory substitution devices such as subcutaneous or external applied tactile stimulation devices to provide useful corresponding output.

### BRIEF DESCRIPTION OF DRAWINGS

Preferred embodiments of the invention will now be described, by way of example only, with reference to the accompanying drawings in which:

FIG. 1A shows the external components of a retinal stimulation system;

FIG. 1B shows the implanted components of the retinal stimulation system of FIG. 1;

FIG. 2 is an illustration of the implanted stimulation electrode array of the system of FIG. 1;

FIG. 3 shows various photographs of the implanted portion of the system of FIG. 1;

FIG. 4 show user wearable glasses for digital image acquisition in the system of FIG. 1;

FIG. 5 is a flow chart of a method according to an aspect of the invention for detecting an object in an acquired image in the system of FIG. 1;

FIG. 6 is an example of implementation of the method of FIG. 5;

FIG. 7 is a flow chart of a method according to an aspect of the invention for determining depth of objects detected in an acquired image in the system of FIG. 1;

FIG. 8 is an example of implementation of the method of FIG. 7; and

FIG. 9 is a schematic illustration of the system of FIG. 1 with externally applied tactile stimulation in place of direct retinal stimulation.

### DESCRIPTION OF EMBODIMENTS

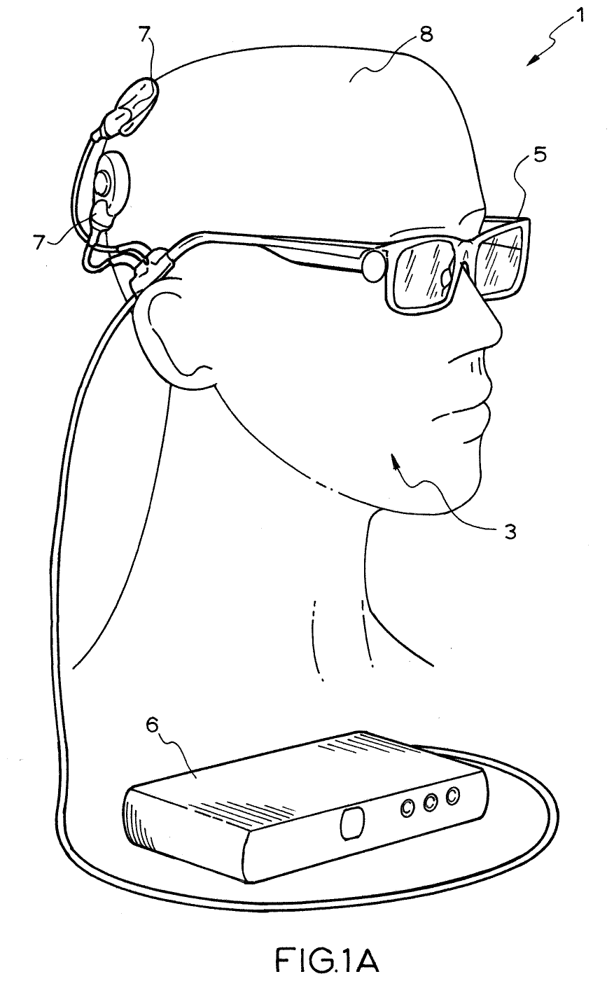

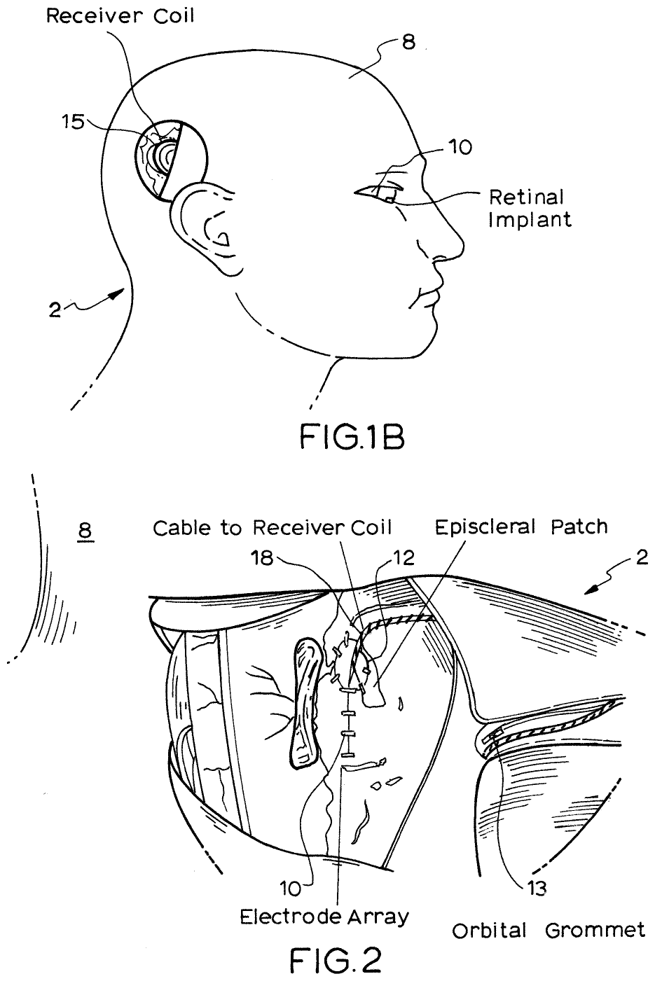

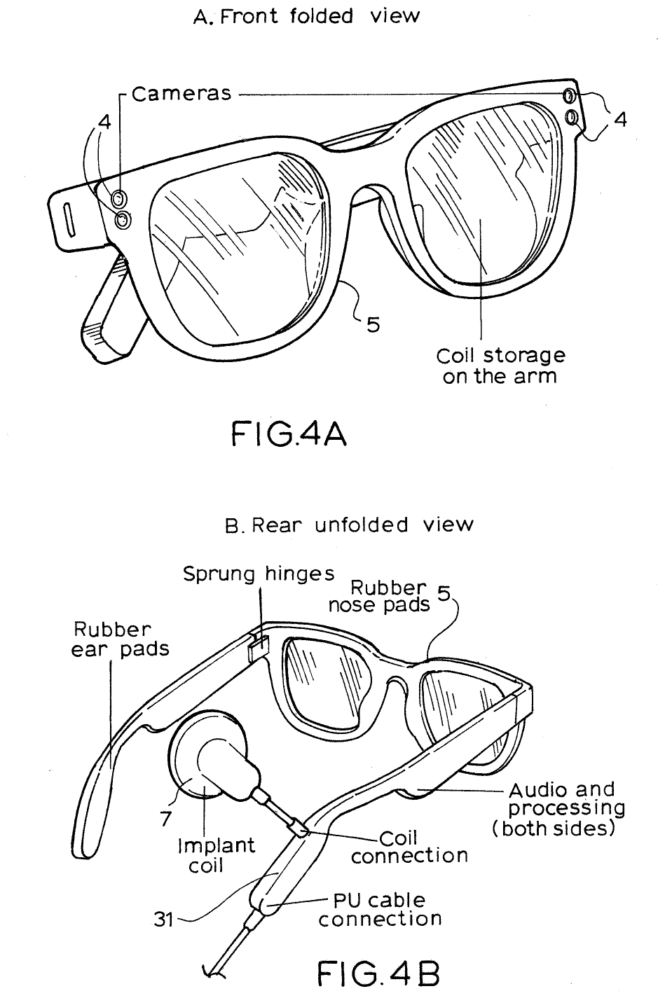

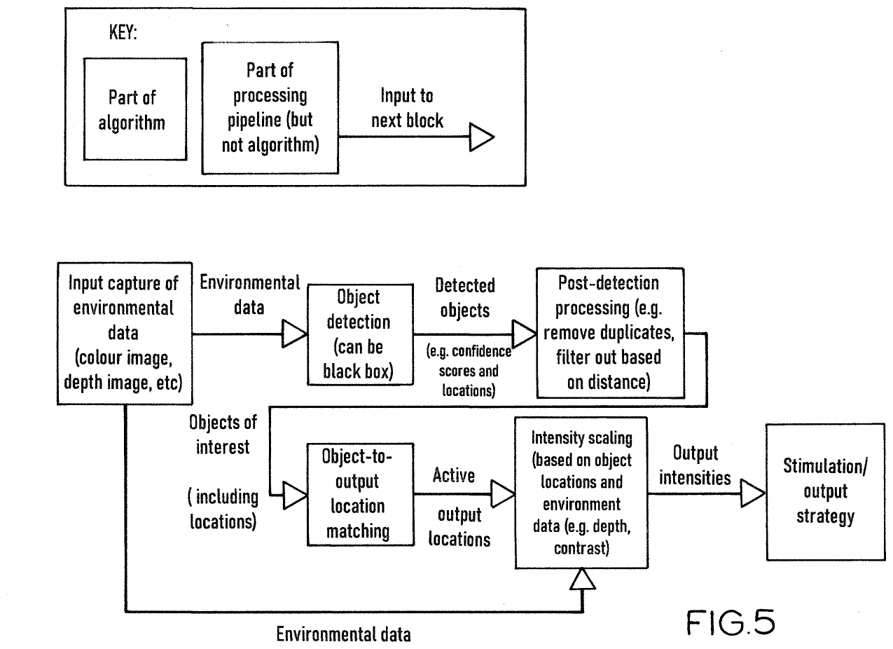

Referring firstly to FIGS. 1 to 4, there is shown a retinal stimulation system 1 comprising implanted 2 and external components 3. The system 1 includes a plurality of digital image acquisition devices in the form of digital camera elements 4 mounted to headset glasses 5. The camera images are sent to an external controller device 6 (denoted PU) for processing including application of the method steps of the preferred embodiment shown in FIGS. 5 & 7 respectively.

Subsequent to processing, signals are sent to one or more induction coils 7 disposed on the outside of a user's head 8 proximal to an implanted receiver coil 15. Corresponding signals are then sent to stimulation electrodes 10 implanted to stimulate the retina, described below, and simulate the position of objects in the camera 4 field of view.

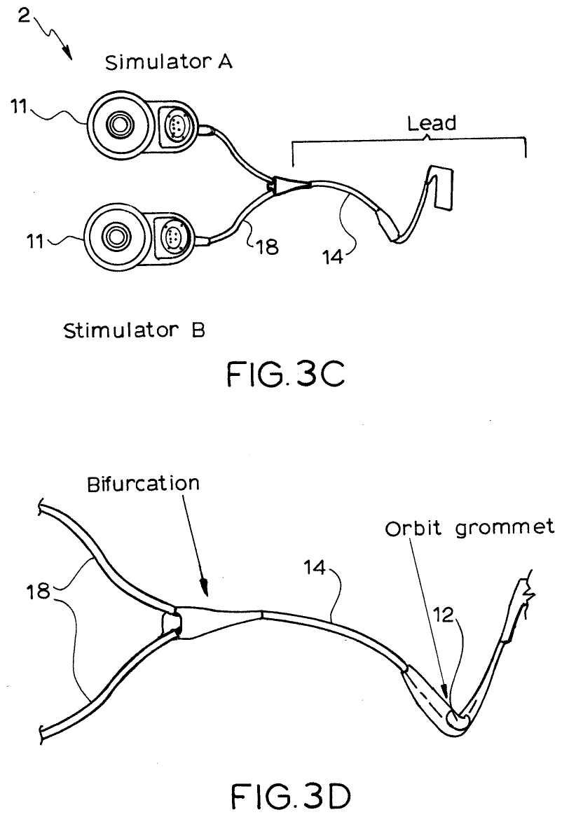

As best seen in FIG. 3, there are three main components of the implant:
The stimulator 11: decodes stimulation commands and drives the stimuli.
The electrode array 10: implanted in the suprachoroidal space to deliver stimuli to the retina.
The lead 14: transfers stimuli from the stimulator to the electrode array.

The stimulation implant can of course be provided in left or right eye configurations which will determine the direction of the bend in the lead between the electrode array 15 and the receiver coil 15.

The stimulator or implanted portion consists of a receiver coil 15, a magnet 16, a stimulator body 17, a return electrode 21, and a lead connection 18 as shown in FIG. 3. The receiver coil 15 inductively couples to the coil 7 on the headset 5 to receive power and data and communicates back to the external PU 6 via the headset 5. The magnet 16 holds the headset coil 7 in alignment. The stimulator body 17 houses electronics that receive the power and data from the coil 15, decode the signal commands, and drive the stimulation current. The stimulator body 17 is hermetically sealed and protects the electronics from environmental factors such as impact and fluid ingress.

A return electrode 21 on the stimulator 10 acts as a return path for the stimulation driven via the electrode array 10. The lead connector 23 connects the lead wires from the electrode array 10 to the electronics inside the stimulator body 17. Body 17 is also hermetically sealed and provides strain relief.

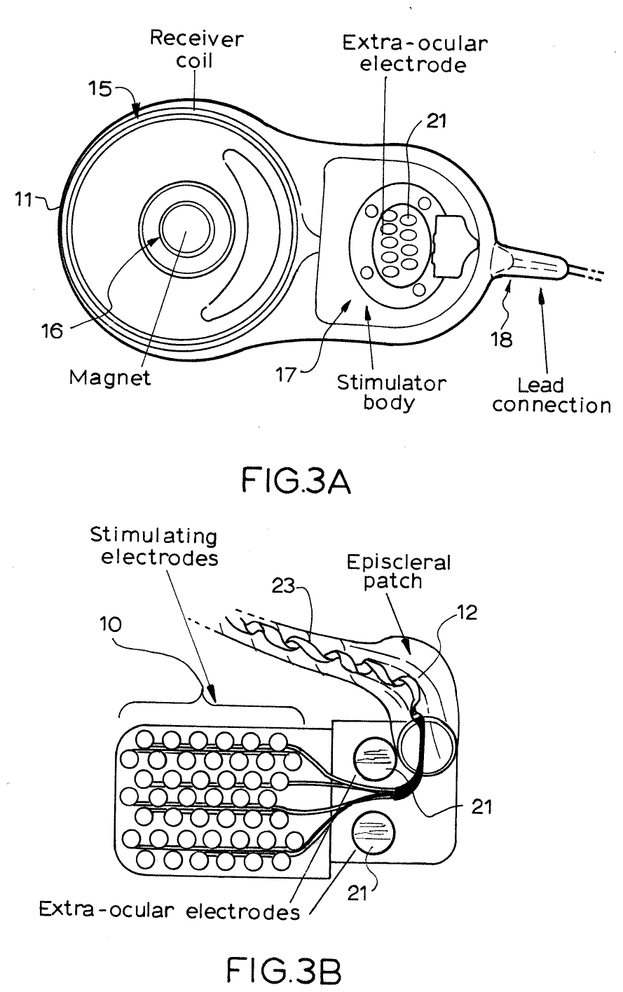

In the system of the preferred embodiment, the retinal stimulation electrode array 10 consists of 44 stimulating electrodes, two return electrodes 21, and an episcleral patch 12. The stimulating electrodes 10 are in a rectangular-grid array and are configured to deliver predetermined stimulation to the retina (best seen in FIG. 2). The return electrodes 21 on the electrode array are used as a second option for a return path for the stimuli delivered via the stimulating electrodes 10 on the electrode array implanted in the suprachoroidal space. The episcleral patch 12 is sutured to the sclera during placement of the electrode array 10 to keep the array from migrating.

Advantageously, having two options (the return electrode on the stimulator and the return electrodes on the electrode array) provides redundancy in the design in case one of the return paths is not able to be used.

The implant lead 23 connecting the stimulator 11 to the electrode array 10 and consists of a number of individual lead wires for each of the stimulating electrodes 10, and for each of the return electrodes 21 on the electrode array. The individual lead wires incorporated into the implant lead form the electrical connections between the electrode drivers in the stimulator 11 and the electrodes in the electrode array 10/21. As best seen in FIG. 2, the implant lead 18 is tunneled along the temporal region of the skull to the orbit where the electrode array 10 is implanted.

It is noted that the retinal stimulation electrodes are surgically placed using a custom trocar (not illustrated). The lead and the electrode array are placed in the custom trocar, then the trocar containing the electrode array is tunneled through the temporal region of the skull to the lateral canthus. The stimulator is placed on top of the trocar while the electrode array is being tunneled. After tunnelling the trocar, the lead is removed from the trocar which itself is then removed by pulling it through an incision near the orbit.

The retinal stimulator rests in the temporal region of the skull superior to the pinna. A pocket in the suprachoroidal space is made and the electrode array is surgically placed in this space. An episcleral patch 12 is sutured to the sclera to prevent migration. The lead 18 rests in the temporal and orbital regions of the skull between the stimulator and the electrode array, with the orbit grommet 13 fixed in an orbitotomy.

Turning now to the external components 3 of the system 1, in the preferred embodiments the headset has a plurality of spaced apart cameras 4, together with electronics, a coil, a cable, and a small speaker 31 are integrated with a set of glasses frames 5. The cameras 4 contain visual sensors which capture color and depth images in real-time. The electronics aggregate the images captured by the cameras 4 and convert them to a format that the external controller or PU 6 can interpret, to then drive the induction coil 7 for power and signals to the implanted receiver coil 15.

The headset 5 contains small speaker 31 to provide audio feedback to the users, for example if the battery is low. Coil 7 couples to the implant stimulator's coil 15 and transfers power and data between the externals 3 and the implant 2. The headset cable acts as an interface between the external PU 6 and the headset 5. The cable passes captured images to the PU 6 and provides power to the headset 5 electronics.

Although not illustrated, the external PU 6 contains user interface (UI) components, power and data interfaces, and electronics contained within a case. The main electronics inside the PU 6 include a processor (including but not limited to CPUs, GPUs, TPUs, Visual Processing Units as well as ASICs and FPGAs), memory, and several interfacing components including Wi-Fi and an audio driver. Preferably, the UI components include volume control buttons, mode selection buttons, a power switch, a status button, and a speaker and PU ports include a headset connection port and a charging port.

The processor controls communications with the headset 5 and also powers the headset. The PU 6 is preferably powered by a non-removable rechargeable battery/s. The PU case houses the electronics and all the buttons, sockets, and batteries. The PU is configured to operate while connected to a portable battery pack, so that it can be recharged while being used. In use, the PU 6 provides an audible notification when the PU battery is low.

In respect of the retinal implant device of the preferred embodiment, this was constructed using commercially available components including the implantable receiver coil stimulators and a single Intraocular Electrode array. Materials used in the implanted device complied with applicable regulatory standards.

Various methods of processing visual information and other environmental data and translating them into a representation of the visual field that can be used to generate a stimulation signal for the retinal prosthesis (or other sensory substitution device or neurostimulation device). The processing steps are intended to provide the user of the system with only relevant information to account for the reduced bandwidth of information that may be present in such a system caused by the limited number of implanted stimulation electrodes.

Once a digital images and sensor data were captured, the data goes through processing steps identify areas of the visual field that are selected or are of interest, prioritise the importance of particular parts of the data, and to filter out parts of the data that are not relevant.

The processing methods in preferred embodiments may have discrete modes that change the parts of the data that are deemed important or are prioritised.

Specifically, the processing steps include: Object identification; Depth filtering to only consider objects within a certain proximity; Applying a method of identifying and ignoring any ground/floor/roof/walls/other planar sections or the image, or other predetermined uninteresting parts of an image; Determining the state of an object based on other objects detected (e.g. determining if a chair available to sit in based on whether other objects such as people are in close proximity to the chair); and Changing between one or more of the above processing steps.

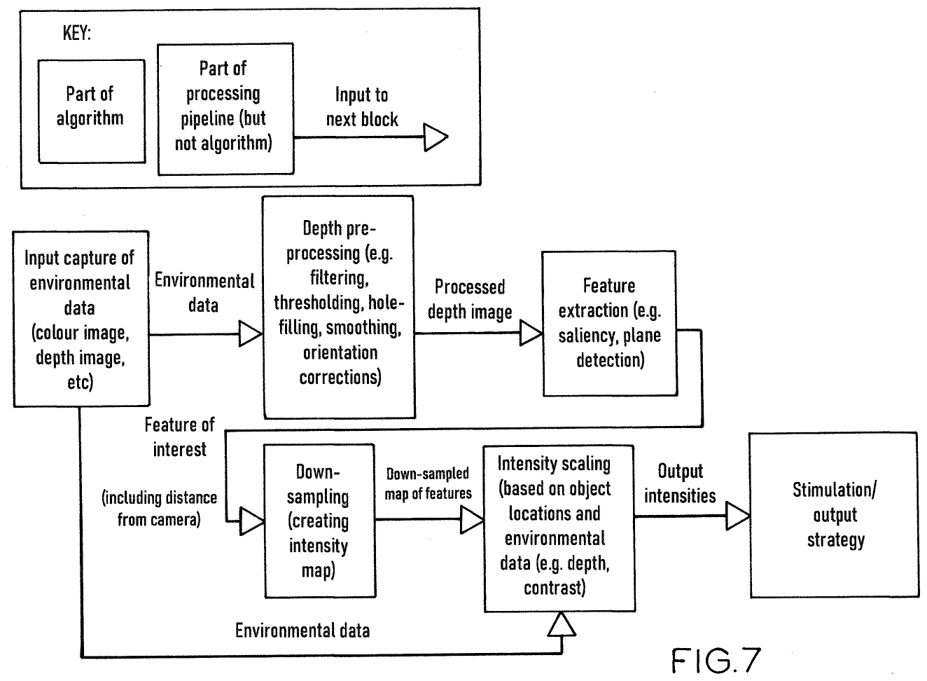

There are two main processing methods included in the preferred embodiment namely, a method of objection detection (FIG. 5) and a method of depth detection (FIG. 7). The object detection algorithm is set out schematically in FIG. 5. In particular these steps are:

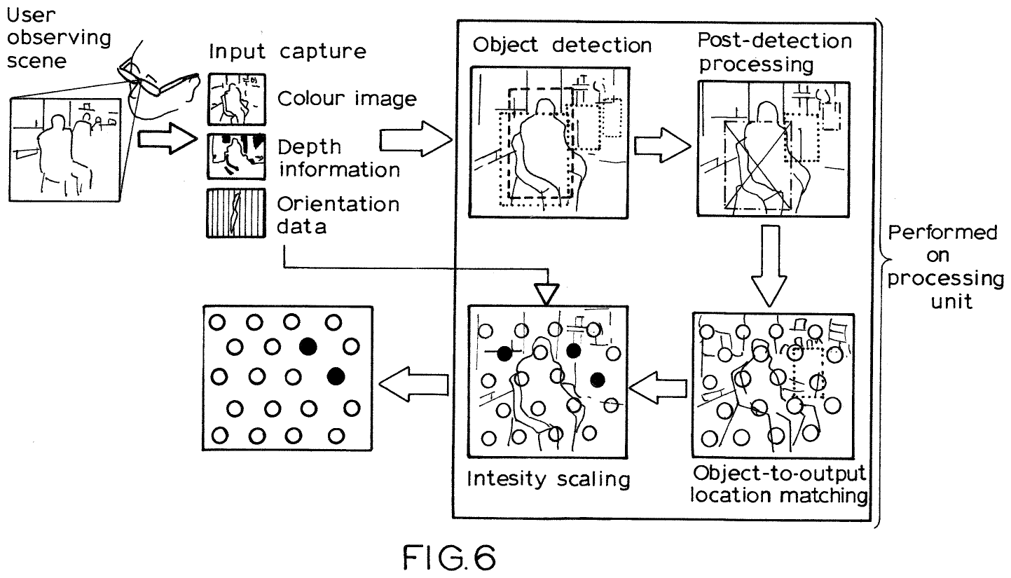

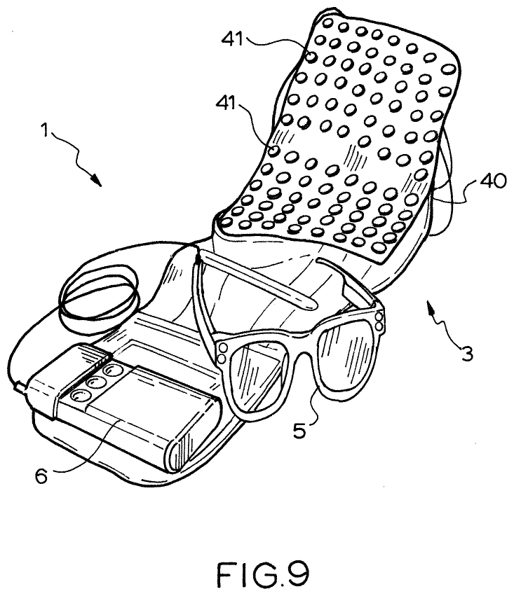

1. Object detection: This step can utilise any specific object detector. Examples include convolutional neural network models such as in the preferred embodiment or non-CNN algorithms such as Haar cascade. These can be selected for accuracy, speed and type of objects to be detected. For example, a YOLO CNN can be employed when processing speed is at issue. The output of this stage can be any kind of location output. Examples include bounding box (as in the embodiment of FIG. 6), centre point and pixel mask amongst others.
2. Post-detection processing: This step cleans or conditions that output of the object detector by removing potential duplicated detections and filtering out detections that are too far away. Advantageously, bandwidth is not used for objects not practically relevant to the user within a predetermined distance in the surrounds.
It can also remove objects that overlap with one another or make inferences about the state of an object based on the detections. For example, it may remove a detected chair if there is a person, handbag, or another object of interest in closed proximity to the chair. This type of filtering can be based on any of the environmental data (e.g. depth proximity, or colour matching), or it can be based on the output of the object detector (e.g. intersection-over-union).
3. Object-to-output location matching: This step maps the detected objects to the location of an output (e.g. a particular electrode or ‘phosphene’ in prosthetic vision, or a tactor such as shown in FIG. 9). The location matching utilised the post-detection objects of interest and correlated them to an output location.
4. Intensity scaling: This step determined the intensity of each electrode output. The intensity scaling was determined by preferred environmental data. However, it could be determined by the contract between the object and its background, or it could be determined by the distance of the object to the user.
5. The output intensities are then passed on to the next stage of the processing pipeline where they will eventually provide an output for use in providing retinal electrode stimulation to the user.

Turning now to the method of determining depth, FIG. 7 schematically sets out the method steps of the depth processing of the digital image of the preferred embodiment:

1. Pre-processing: This step cleans or conditions the environmental data and includes temporally and spatially smoothing the data to reduce artefacts, filling holes in the data, determining the orientation of the data in 3D space and filtering the data to remove unwanted elements, amongst others.
2. Feature extraction: This step identified key features in the image such as the ground plane, salient objects, or shorelines using the processed environmental data.
3. Down-sampling: This step creates an intensity map with a reduced resolution compared to the original environmental inputs. This reduced the likelihood of flickering or spurious outputs subsequently. Down-sampling can be performed using any method. Some examples include Voronoi mapping around output locations as in the preferred embodiment, and Gaussian sampling.
4. Intensity scaling: This step determined the intensity of each output for each electrode or phosphene. The intensity scaling can be determined by predetermined environmental data in combination with the feature extraction and/or down-sampled intensity map. For example, it could be determined by the contract between the object and its background, or it could be determined by the distance of the object to the user.
5. The output intensities were then passed on to the next stage of the processing pipeline where they will eventually form part of an output to the user in the form of electrode stimulation.

Most advantageously, the system enables changing the active mode between these methods (and/or others also), including different instances of the algorithms implemented with different parameters. This may be done in two ways, the first being via user input/selections where the user provides an input to the system to determine which mode should be activated. For example, pressing a Bluetooth button, or a button on the PU housing or glasses frame, or issuing a voice command. The second is via an automated changing.

In the second option (automated changing), the system PU analyses the environmental inputs and changes mode or optimises algorithm parameters. For example, the system may determine that there are no objects in close proximity, so it increases a depth filtering threshold. Or the system may identify that there are no faces in the scene, but there are chairs in the scene, so it changes mode to identify chairs. Objects of importance to the user such as chairs, faces, doorways or handles, keys or any other object can be predefined to be selectable.

It will be appreciated that a state of a detected object can be determined based on any pre-selected states. In the case of the preferred embodiments, the chair once detected and identified may have its state (as noted elsewhere) detected including whether it is occupied or obstructed; and an emotional state of the face could be detected. The system may alert the user when this occurs. Any kind of alert may be used. For example, a vibration or an auditory cue such as spoken alerts as used via the speaker in the preferred embodiment.

The processing of information from the real world into stimulation of the user follows the sequence of image capture; data processing; and application of stimulation. In the image capture step, the glasses worn by a user have multiple on-board cameras and/or other sensors as noted above and these other sensors can include, for example, gyroscopes, accelerometers and thermal sensors. These cameras and sensors capture different information about the environment in front of the patient. For example, the preferred embodiment employs a standard colour sensor, two infra-red sensors and a gyroscope/accelerometer sensor.

The combination of various sensors provides the system with information such as colour, colour contrast, depth/distance to objects, orientation with respect to the environment (e.g. facing up or turned sideways), and changes in orientation. It is noted for any given moment, this data is collectively referred to as a “frame”. Each time a capture was performed the frame data was passed to the external Processing Unit for data processing.

In the data processing step, the data processing capability of the external PU is implemented in software as a series of data processing stages termed a pipeline. The pipeline consists of a series of stages that take a frame as input and progressively process the frame in order to output a sequence of instructions that can be interpreted as a stimulation sequence/pattern (“output intensities”) for the electrodes of the implanted array.

Described below, the preferred embodiment combines both object and depth detection. That is, a pipeline of operations that starts by performing image data processing to clean up or condition any input frames, followed by object detection on that data. An example of this is where the depth information in a given frame is suboptimal, image processing algorithms are first run to provide a clean/more accurate depth image, followed by object detection to identify a chair (in the preferred embodiment but this can be any desired object) that is within the patient's field of view and reach.

However, in other preferred embodiments, the object detection and depth methods can operate independently. An example of this is where the depth information in a given frame is suboptimal, image processing algorithms are first run to provide a clean/more accurate depth image and then the information about everything within a predetermined or allowable depth distance is presented to the user through stimulation.

Referring particularly to the object detection method of FIG. 5, an end-to-end implementation of the preferred embodiment includes the following steps, resulting in the stimulation output shown in FIG. 7.

Colour images, depth information and orientation data are captured by the set of glasses worn by the user. This data is passed on to the processing unit.

The processing unit runs the object detection method to find chairs and people as defined objects in the preferred embodiment.

Post-detection processing removes chairs in close proximity to people. It also removes chairs that are too far away. That is, detected objects are removed when displaying them offers little practical benefit.

The output locations are mapped to the chairs that have not been removed.

The depth data is used to scale the output intensity in one of the following ways (these are only examples used in the preferred embodiments):
a. All electrodes or phosphenes that are within the bounding box of the chair are activated according to a statistical analysis of the depth values inside the bounding box of the chair (e.g. the mean, median, percentile). Phosphenes just outside the bounding box may also be activated to draw the user's attention to an object. The advantage of this method is that the user receives a very clear signal of the chair. This is the method used in the images of the flowchart of FIG. 5.
b. One phosphene may be activated for each chair (e.g. the phosphene closest to the centre of the chair) according to a statistical analysis of the depth values within the bounding box. The advantage of this method is that enables the user to count the number of chairs in a scene.
c. The phosphenes are scaled according to their distance from the centre of the object. E.g. a Gaussian disc was used in the preferred embodiment around the centre of the chair, and the closer to the centre, the brighter the phosphene will be. The advantage of this method is that is provides better acuity and localisation of applied stimulation for the user.

The stimulation output values are determined and passed on to the output device (the external PU 6 to the implant in this embodiment).

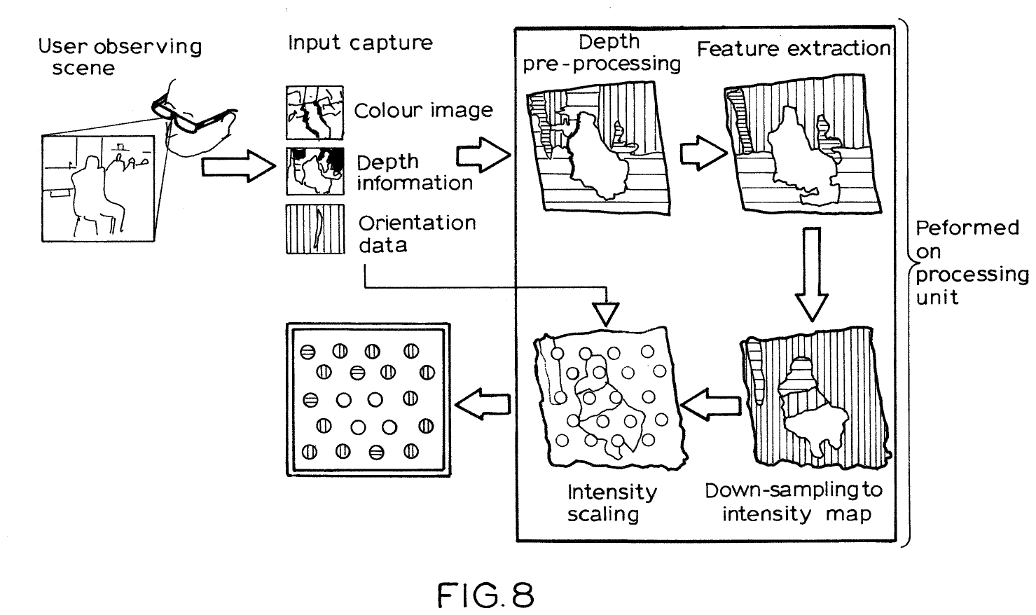

Referring now to the depth detection method of FIG. 7, an end-to-end implementation of the preferred embodiment includes the following steps, resulting in the output shown in FIG. 8.

Colour images, depth information and orientation data are captured by the set of glasses worn by the user. This data is passed on to the processing unit.

The processing unit controls the following pre-processing steps:
a. Hole-filling: small gaps in the depth image were filled using a method which made any undefined value equal to the value to the left of it. This can cascade across the depth map. It will be appreciated any preferred method can be used.
b. Smoothing: both temporal and spatial smoothing were used to remove noise in the depth map.
c. Thresholding: depth values beyond a predetermined range were removed to limit the range of influence on the output.
d. Orientation correction: the depth value was used to generate cartesian coordinates (x-y-z), so that an absolute frame-of-reference was used in the depth processing further down the pipeline. This advantageously eliminated any issues related to the orientation of detected planes.

The processing unit then extracted features from the pre-processed depth map. The features included:

a. Edges: contours that represented the edge of an object or wall. This allowed users to follow a wall, or more clearly identify an object.
b. Ground plane: this was identified so that it could be ignored in the output. Users do not have an interest in the ground, and ignoring it allows better detection of low-lying obstacles and trip hazards. Examples of ground plane detection algorithms include (but are not limited to):
i. RANSAC dominant plane detection.
ii. Deep-learned models trained for ground detection.
iii. Histogram analysis of y-coordinates.
   c. Salient objects: objects that stick out from the background.

An intensity scaling map was then created based on the features. Ground was ignored. Edges and salient objects were highlighted and scaled according to the distance from the user. The intensity map was used to scale the output intensity in one of the following ways (these are only examples):

(a) A Voronoi region around each electrode or phosphene was defined. A statistical value within the region was used to determine the output of the phosphene (e.g. max, median, percentile); or (b) a disc was created around the phosphene with a scaling function (e.g. gaussian). The value of the intensity as based on the maximum or average scaled value within that disc. This method allowed for greater acuity. This is the method used in the images of FIG. 8.

The stimulation output values were determined and passed on to the output device (the implant in this embodiment).

Referring lastly to FIG. 9, there is shown another implementation of the methods of the preferred embodiments. This shows a system 1 identical to FIG. 1, however, the stimulation output is not provided to the implanted electrode array 10 on the retina but to an array of ‘tactors’ 40 disposed in similar preferred grid pattern but which are placed in direct (on) or indirect (clothing intermediate) with the skin of a person to receive the ‘mapped’ image in that manner.

Here, the intensity is provided via a vibrational force, electrical stimulation or application of hot/cold. The preferred embodiment uses a patch 41 having an array of tactile stimulators 40 where the patch 41 attaches to the chest or back of the user. A stimulation map is provided in the same manner with the advantageous object detection &/or depth processing algorithms but also that no surgery or implantation is necessary. Of course, tactile stimulation is limited to the resolution a user can make between tactors however a reliable and accurate map is still provided to the user.

It will be appreciated that in other preferred embodiments of the invention (not illustrated) the object detection method and/or the depth processing method can be used with any visual sensory substitution device whether implanted retinal stimulation, external tactile stimulation or direct neural stimulation (eg to the cerebral cortex).

The foregoing describes only one embodiment of the present invention and modifications, obvious to those skilled in the art, can be made thereto without departing from the scope of the present invention.

The term “comprising” (and its grammatical variations) as used herein is used in the inclusive sense of “including” or “having” and not in the exclusive sense of “consisting only of”.

1. A method of operating a visual sensory substitution device, the method comprising the steps of:
receiving image data and other data indicative of an externally generated image representative of environmental data;
detecting one or more predetermined objects from the image data;
processing the post-object detected image data;
matching the location of detected objects to corresponding locations of the stimulation device;
scaling the intensity of output to the corresponding locations of the stimulation device;
providing output data for use by the stimulation device representative of the location of the detected objects.

2. A method according to claim 1 wherein the image data includes colour data, depth data, contrast data and intensity data.

3. A method according to claim 1 including the step of ascribing confidence scores to the one or more detected objects.

4. A method according to claim 1 wherein processing the post-object detected data includes the step of identifying and removing duplicate detected objects, and filtering the data as a function of distance.

5. A method according to claim 4 wherein processing the post-object detected data includes removing a detected object in response to the detected object being in a defined state.

6. A method according to claim 5 wherein the detected object is a chair, or any other type of object that can be used as a seat, and the detected state includes being occupied or unoccupied, or obstructed.

7. A method according to claim 1 wherein the step of scaling the intensity of the output data is dependent on detected object location and environmental data.

8. A method according to claim 7 wherein the environmental data includes depth or distance and contrast.

9. A method according to claim 1 further including the steps of:
receiving image data and other data indicative of an externally generated image representative of environmental data;
depth pre-processing the received image;
extracting predetermined features from the pre-processed image;
down-sampling the pre-processed image to create a corresponding intensity scaling map of the intensity of corresponding to the location stimulation device,
scaling the intensity of the pre-processed image using the intensity scaling map; and
providing output data for use by the stimulation device.

10. A method according to claim 9 wherein the image environmental data includes colour data, depth data, contrast data and intensity data.

11. A method according to claim 9 wherein depth preprocessing includes one or more of: temporally or spatially smoothing the received image data to remove or minimise artefacts; determining an orientation of the image data and removing any predetermined undesirable data; hole filling; thresholding; and orientation corrections.

12. A method according to claim 9 wherein the extracted predetermined features include salient objects, detected planes, shorelines and distance.

13. A method according to claim 9 including scaling the intensity as a function of object or feature location and associated environmental data.

14. A method according to claim 1 wherein the sensory substitution device is a retinal implant having an array of stimulating electrodes.

15. A method according to claim 1 wherein the sensory substitution device is a tactile sensor having a plurality of spaced apart tactors, the tactile sensor configured to be disposed contiguous with or closely adjacent skin of a user.
